(19) United States
(12) Patent Application Publication
(10) Pub. No.: US 2020/0129145 A1
(43) Pub. Date: Apr. 30, 2020

(54) INTRAVASCULAR ULTRASOUND CATHETER SYSTEMS AND METHODS FOR USING THE SAME

(71) Applicant: Abbasi Vascular Solutions, Inc., Flint, MI (US)

(72) Inventor: Mohammad Nabeel Abbasi, Grand Blanc, MI (US)

(73) Assignee: Abbasi Vascular Solutions, Inc., Flint, MI (US)

(21) Appl. No.: 16/665,097

(22) Filed: Oct. 28, 2019

Related U.S. Application Data
(60) Provisional application No. 62/751,145, filed on Oct. 26, 2018.

(51) Int. Cl. A61B 8/08; A61B 8/12; A61B 8/00; A61B 8/04; A61M 25/00

(52) U.S. Cl. CPC A61B 8/0891; A61M 2205/0294; A61B 8/445; A61B 8/4494; A61B 8/466; A61B 8/04; A61M 25/0045; A61M 25/0068; A61M 25/0014; A61M 2025/0002; A61M 2025/0057; A61M 2205/0238; A61M 2205/0244; A61M 2205/3368; A61B 8/12

(57) ABSTRACT

An intravascular ultrasound (IVUS) catheter or wire for imaging body tissue, including a catheter body having a proximal end, a distal end opposite thereto, and an outer surface having a plurality of ultrasound transducers operatively attached thereto that provide real-time and continuous imaging of the body tissue along an entire length of the body tissue. A method of using an IVUS catheter or wire for imaging tissue in real-time, by inserting an IVUS catheter in the body of an individual, wherein the IVUS catheter includes a catheter body having a proximal end, a distal end opposite thereto, and an outer surface having a plurality of ultrasound transducers, and providing real-time and simultaneous imaging of tissue with the plurality of ultrasound transducers. Methods of performing trans cardiac echocardiography, trans esophageal echocardiograph, imaging the bladder and prostate, and imaging the gastrointestinal system.

### BACKGROUND OF THE INVENTION

1. Technical Field

The present disclosure relates generally to catheters and more particularly to intravascular ultrasound catheters having multiple ultrasound transducers.

2. Background Art

Intravascular ultrasound (“IVUS”) is a technology used to generate ultrasound images of parts of the vascular system. IVUS is performed using a catheter that includes a miniaturized ultrasound probe positioned at its distal end. IVUS imaging may be useful in a variety of procedures, for example, imaging the shapes and thicknesses of tissues such as vascular plaque and vessel walls, determining the diameter of vessel lumen, and determining if stents have been fully opened during deployment within the vascular system.

Existing IVUS catheters have been used predominantly for diagnostic purposes. Present IVUS imaging techniques produce approximately ten images of the blood vessel per second while a user e.g., vascular surgeon, interventional cardiologist, or any other suitable practitioner) slowly retracts the IVUS catheter through the vessel. These images are compiled together to produce a two-dimensional (2-D) representation along a length of the vessel called a pullback image, with a typical maximum field of view diameter of six centimeters. The images produced using existing IVUS catheters may contain motion artifacts. Further, the 2-D representation of the vessel may contain errors since the images used to generate the 2-D representation were taken at discrete moments during the vessel's dilation and constriction. Additionally, the 2-D representation is a snap shot of the vessel at one point in time and does not give a continuous live view of the entire length of the vessel.

For at least the above reasons, existing IVUS catheters are limited in their current use for therapeutic interventions. Some are using IVUS guided procedures but are doing this by using angiography in combination to mark the positions of the catheter at the point of interest, again this is a snap shot and a continuous live image.

Angiography is currently used in therapeutic cardiovascular procedures. Angiography provides a way to image the blood flow within a vessel by injecting a radiopaque contrast dye within a vessel and taking x-ray images of the resultant blood flow. However, disadvantages to angiography include exposing the patient to potentially harmful x-ray radiation, and risking damage to the patient's kidneys from the use of nephrotoxic contrast dye. Further, only the radiopaque dye, and thus the geometry of the lumen, is visible during an angiogram. Information about the plaque thickness or vessel wall geometry is not known from using angiography. Thus, a need exists for IVUS catheters that can be used in therapeutic treatment of cardiovascular or other vascular disease.

### SUMMARY OF THE INVENTION

The present invention provides for an intravascular ultrasound (IVUS) catheter and/or wire for imaging body tissue, including a catheter body having a proximal end, a distal end opposite thereto, and an outer surface having a plurality of ultrasound transducers operatively attached thereto that provide real-time and continuous imaging of the body tissue along the entire length of the body tissue or vessel in question.

The present invention provides for a method of using an IVUS catheter for imaging tissue in real-time, by inserting an IVUS catheter or wire in the body of an individual, wherein the IVUS catheter includes a catheter and or wire body having a proximal end, a distal end opposite thereto, and an outer surface having a plurality of ultrasound transducers, and providing real-time and simultaneous imaging of tissue with the plurality of ultrasound transducers along the entire length of the tissue.

The present invention provides for a method of performing trans cardiac echocardiography by inserting an IVUS catheter and/or wire in the body of an individual through jugular, femoral or subclavian venous access and then subsequently guiding the device into the individual's right atrium, right ventricle or pulmonary artery, wherein the IVUS catheter and or/wire includes a catheter body having a proximal end, a distal end opposite thereto, and an outer surface having a plurality of ultrasound transducers, and providing real-time and simultaneous imaging of the individual's heart with the plurality of ultrasound transducers and allowing for imaging of the cardiac chambers in real time.

The present invention provides for a method of performing trans esophageal echocardiography by inserting an IVUS catheter in the body of an individual into an esophagus, wherein the IVUS catheter includes a catheter body having a proximal end, a distal end opposite thereto, and an outer surface having a plurality of ultrasound transducers, and providing real-time and simultaneous imaging of the individual's heart with the plurality of ultrasound transducers throughout the entire length of the device.

The present invention provides for a method of imaging the bladder and prostate by inserting an IVUS catheter and or/wire in the body of an individual transurethrally, wherein the IVUS catheter and or/wire includes a catheter body having a proximal end, a distal end opposite thereto, and an outer surface having a plurality of ultrasound transducers, and providing real-time and simultaneous imaging of the individual's bladder and prostate with the plurality of ultrasound transducers along the entire length of the device.

The present invention also provides for a method of imaging the gastrointestinal system by inserting an IVUS catheter in the body of an individual into the gastrointestinal system, wherein the IVUS catheter includes a catheter body having a proximal end, a distal end opposite thereto, and an outer surface having a plurality of ultrasound transducers, and providing real-time and simultaneous imaging of the individual's gastrointestinal system with the plurality of ultrasound transducers along the entire length of the device.

### BRIEF DESCRIPTION OF THE DRAWINGS

Reference will now be made to exemplary embodiments, examples of which are illustrated in the accompanying drawings. Wherever possible, the same reference numbers will be used throughout the drawings to refer to the same or like parts. The drawings are not necessarily to scale.

FIG. 1A illustrates a perspective view of a treatment system comprising an intravascular ultrasound (IVUS) catheter and a user interface module according to various embodiments of the present disclosure, and FIG. 1B shows the IVUS wire being used as a transducer.

FIG. 2 is a cut-away view of a blood vessel with the catheter as shown in FIG. 1 positioned therein, according to various embodiments of the present disclosure.

FIG. 3 illustrates the catheter and blood vessel shown in FIG. 2 with the catheter and blood vessel displayed in a uncurled configuration.

FIG. 4 illustrates one embodiment of a user interface layout depicting an ultrasound image of an axial cross-section of a blood vessel.

FIG. 5 illustrates one embodiment of a user interface layout depicting an ultrasound image of a longitudinal cross-section of a blood vessel.

FIG. 6 illustrates one embodiment of the functionality of a user interface layout, according to various embodiments of the present disclosure.

FIG. 7 illustrates an exemplary three-dimensional (3-D) reconstruction of blood imaged according to various embodiments of the present disclosure.

FIG. 8 illustrates use of the IVUS catheter/wire in performing trans cardiac echocardiography.

### DETAILED DESCRIPTION OF THE INVENTION

The present invention provides generally for IVUS catheters and/or wires that can provide images continuously along the entire length of the device allowing for real time visualization of the body or tissue in question throughout the length of the device as opposed to current devices which can only image at one point of imaging at a time. While the present invention is especially useful in imaging blood vessels, it should be understood that the catheters or wires herein can be used to image any part of the body or tissue desired. While reference is made herein to a “catheter”, it should be understood that a “wire” can have the same structure and function.

Reference will now be made in detail to various embodiments of the disclosed devices and methods, examples of which are illustrated in the accompanying drawings. Wherever possible, the same reference numbers will be used throughout the drawings to refer to the same or like parts.

In the present disclosure, the use of the singular includes the plural unless specifically stated otherwise. In the present disclosure, the use of “or” means “and/or” unless stated otherwise. Furthermore, the use of the term “including” as well as other forms, such as “includes” and “included,” is not limiting. Any range described herein will be understood to include the endpoints and all values between the endpoints.

The section headings used herein are for organizational purposes only and are not to be construed as limiting the subject matter described. All documents, or portions of documents, cited in the present disclosure, including but not limited to patents, patent applications, articles, books, and treatises, are hereby expressly incorporated by reference in their entirety for any purpose.

Embodiments of the present disclosure relate to IVUS catheter and/or wire systems and methods used to image tissues within the body, such as blood vessels and arterial plaques, the heart and cardiac chambers, the gastrointestinal system, or the genitourinary system. The IVUS systems and methods disclosed herein can allow users (e.g., a vascular surgeon, interventional cardiologist, or other suitable healthcare practitioner) to image tissues continuously and/or in real-time and use this to guide or deliver therapy such as stenting, angioplasty, heart failure therapy, or transurethral resections.

Further, embodiments of the present disclosure enable a user to administer both diagnostic and therapeutic treatment to a patient. In some embodiments, IVUS catheter systems and/or wires of the present disclosure achieve the above-mentioned medical benefits by analyzing data from multiple ultrasound transducers (or groups of ultrasound transducers) positioned within the device.

Embodiments of the present disclosure relate to image processing techniques that result in improved imaging capabilities, as compared to existing systems. For example, software algorithms can be used to combine data transmitted from multiple ultrasound transducers positioned at various locations along the catheter or wire to produce three-dimensional renderings of the tissues at an area of interest in real time.

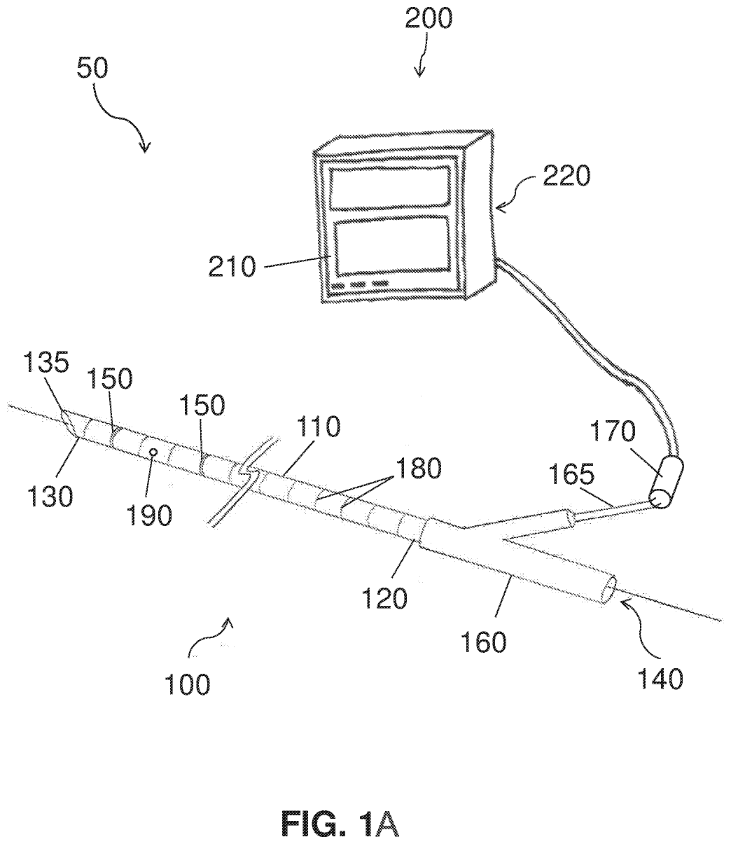

FIG. 1A illustrates a perspective view of a treatment system 50 comprising an IVUS catheter 100, user interface module 200, according to various embodiments of the present disclosure. As shown in FIG. 1A, IVUS catheter 100 includes a catheter body 110. Catheter body 110 has a proximal end 120, a distal end 130, an outer surface 136, and a length L extending between proximal end 120 and distal end 130. FIG. 1B shows an IVUS wire 100 as a transducer.

According to various embodiments, catheter and/or wire body 110 can be a flexible material such as a biocompatible polymer, elastomer, silicon, nylon, combinations of desirable materials, or any suitable biocompatible material. In various embodiments, the material of catheter body 110 includes at least one of polyurethane, polyethylene, polyvinylchloride, polytetrafluoroethylene, or nylon.

The materials of catheter and/or wire body 110 can be selected to produce desired mechanical, biologic, and/or chemical properties. For example, the materials can be selected to allow a desired stiffness/flexibility, to prevent undesired chemical reaction with physiologic fluids, or to resist or prevent infection, thrombus formation, or other adverse clinical consequences.

In some embodiments, the surfaces of catheter body 110 can be coated with a hydrophilic coating to reduce friction between catheter body 110 and various organs and tissues while the catheter is manipulated within the patient. In some embodiments, catheter body 110 can include a heparin-based or other anti-thrombotic coating to prevent blood clotting in and around the device during use.

According to various embodiments, catheter body 110 is provided in a variety of sizes and configurations to suit patients of various sizes and anatomies. For example, catheter body 110 can be provided in lengths that measure about 10, 20, 30, 40, 50, 55, 60, 65, 70, 75, 80, 85, 90, 95, 100, 105, 110, 115, 120, 125, 130, 135, 140, 145, or 150 cm. These values may be used to define discreet lengths, such as 100 cm, or ranges of lengths, such as 105-115 cm.

Additionally, catheter body 110 can have a variety of diameters, defined in medicine using the French (Fr) scale. The units in the French scale range from 3 to 34 and are equivalent to the diameter of a catheter, in millimeters, multiplied by 3. In some embodiments, catheter body 110 can be 3, 3.5, 4, 4.5, 5, 5.5, 6, 6.5, 7, 7.5, 8, 8.5, 9, 9.5, 10, 10.5, 11, 11.5, or 12 Fr. These values can be used to define discrete diameters, such as 7.5 or 9 Fr, or ranges of diameters, such as 6-8 Fr. In an exemplary embodiment, the diameter of catheter body 110 is 8 Fr.

The IVUS wire 100 can also come in various lengths and diameters. The wire can be available in 180 cm, 260 cm, 300 cm or 400 cm lengths. Additionally the wire can be available as a 0.014″ diameter, 0.018″ diameter, or a 0.035″ diameter.

In various embodiments, IVUS catheter 100 also includes at least one lumen 140 extending within and at least partially along the length of catheter body 110. Lumen 140 may be provided in various quantities, sizes, shapes, and lengths. For example, in some embodiments, IVUS catheter 100 can include 1, 2, 3, 4, 5, 6, 7, 8, 9, or 10 lumen. Additionally, in some embodiments, IVUS catheter 100 can include lumens with various diameters, including about 0.05, 0.1, 0.2, 0.3, 0.4, 0.5, 0.6, 0.7, 0.8, 0.9, 1.0, 1.1, 1.2, 1.3, 1.4, 1.5, or 2 mm in diameter.

Lumen 140 can be used for vascular access, drug delivery, sensor containment, access for additional instruments, imaging, and physiologic monitoring. To provide fluid communication or access to the vasculature of a patient, IVUS catheter 100 can further include at least one opening 190 extending between lumen 140 and the exterior of catheter body 110. Opening 190 can vary in quantity, size, shape, and location to accommodate various medical needs. In some embodiments, opening 190 can be defined by a void in the catheter body 110 through which fluid in the lumen can flow into or out of the catheter 110.

In some embodiments, distal end 130 of catheter body 110 can include atraumatic tip 135. Atraumatic tip 135 can be shaped to prevent trauma to surrounding tissues during movement of the IVUS catheter 100, which could be caused by physiological activity, like pulsatile blood flow, catheter manipulation, or user manipulation. Preventing trauma to tissues of the vascular system during medical procedures is vital in avoiding inadvertently creating thrombogenic regions or dissections in the wall of the vessel, which can result in blood clotting and irregular blood flow patterns. Atraumatic tip 135 can be rounded and smooth so that such tissue damage is avoided.

In various embodiments, atraumatic tip 135 of catheter body 110 can include a preformed tip, which can be provided in a variety of configurations, including, but not limited to, C-shape, S-shape, or J-shape. Preformed tips can assist clinicians in maneuvering IVUS catheter 100 through tortuous vessels of the vascular system, such as the internal cavities of main heart and branch vessels.

According to various embodiments, the IVUS catheter 100 can further include connector hub 160 positioned near proximal end 120 of catheter body 110. In some embodiments, connector hub 160 can be a y-connector or manifold connector. Connector hub 160 can bridge catheter body 110 and lumen 140 with one or more access lines 165. In some embodiments, the one or more access lines 165 can serve various functions, for example, providing a conduit for electrical wiring.

In an exemplary embodiment, IVUS catheter 100 can include multiple access lines 165. For example, IVUS catheter 100 can include 2, 3, 4, 5, 6, 7, or 8 access lines 165. Generally, access lines 165 enable a user to perform various functions at a particular region within the body, remotely, like administering medicine or flushing a particular area with saline. In some embodiments, access lines 165 can be distinctly marked or colored to enable users to easily distinguish one access line 165 from another. For example, access lines 165 can be color coded. In some embodiments, access lines 165 can include single-lumen or multi-lumen tubing. In various embodiments, access lines 165 can bifurcate into two additional access lines 165.

In various embodiments, IVUS catheter 100 includes a plurality of ultrasound sensors or ultrasound transducers 150 disposed along a portion of the length of catheter body 110 and operatively attached to the outer surface 136. Ultrasound transducers 150 can transmit and/or receive acoustic data. In various embodiments, IVUS catheter 100 can include 2, 3, 4, 5, 6, 7, or 8 or more ultrasound transducers 150. For example, IVUS catheter 100 can include from 5 to 3000 transducers depending on the spacing constraints of catheter 100. In various embodiments, ultrasound transducers 150 can be provided as microelectromechanical sensors, capacitive sensors, piezoelectric sensors, or a combination therebetween. In some embodiments, ultrasound transducers 150 are provided as capacitive micro-machined ultrasound transducers (i.e., CMUT), which are small form factor MEMS-based devices.

According to various embodiments, ultrasound transducers 150 can be disposed, attached, or secured along or within a portion of catheter body 110, at or near distal end 130. In some embodiments, groups of ultrasound transducers 150 can be positioned at discrete locations along the length of catheter body 110.

In various embodiments, user interface module 200 includes monitor 210 and imaging engine 220. Imagining engine 220 can execute various functions, including data acquisition, data processing, image generation, and data storage. Imagining engine 220 can also display images onto monitor 210. In various embodiments, monitor 210 can include a LCD display, LED display, touch screen, or other suitable means to display ultrasound data (i.e., images) collected by the system 50. In some instances, a user may want to determine certain information about the imaged vessel, such as the density of a plaque or the diameter of its lumen. In these cases, a user may input commands through monitor 210, and imaging engine 220 can perform the required data analysis to calculate and display the requested information.

During data acquisition, user interface module 200 can send an electrical signal to ultrasound transducers 150 disposed along a portion of the length of catheter body 110. This signal can be sent continuously during operation of treatment system 50 to generate real-time, continuous imaging. Once the electrical signal (e.g., high frequency pulse) is transmitted, the ultrasound transducer 150 converts the received electrical signal into an acoustic energy pulse or pressure wave emitted in a 360° manner about catheter body 110.

In various embodiments, when the emitted signals reach tissue to be imaged, they reflect off of the tissue. Then, ultrasound transducers 150 acquire the reflected acoustic signals and acoustic signal (e.g., sound energy) back into an electrical signal (e.g., electrical energy). This electrical signal can then be transmitted back to the user interface module 200, and ultimately to the imaging engine 220 for signal processing and image reconstruction. According to various embodiments, acoustic data can be transmitted between ultrasound transducers 150 and user interface module 200 via a wired or wireless connection.

In some embodiments, IVUS catheter 100 can be operated at various frequencies, such as, for example, 40 MHz or 60 MHz. In some embodiments, IVUS catheter 100 can be operated at a range of frequencies that can vary along the length of the catheter. In some embodiments the frequency range is 20 to 100 MHz. In various embodiments, the frequency range is set for optimal image quality, while maintaining safe levels of exposure to surrounding blood and tissues. In some embodiments, acoustic data is transmitted to user interface module 200 via terminal 170, which may comprise a passive intermodulation (i.e. PIM) connector.

In some embodiments, ultrasound transducers 150 can be spaced apart at set intervals along catheter body 110. For example, the distance between ultrasound transducers 150 can be about 0.5, 1, 2, 3, 4, 5, 6, 7, 8, 9, 10, 15, or 30 mm. For example, the distance between ultrasound transducers 150 can be 0.5, 5, or 10 mm. In an exemplary embodiment, the space between ultrasound transducers 150 results in an ultrasound image with optimized resolution, while minimizing or eliminating cross-signal noise that may adversely affect image quality.

During a minimally invasive medical procedure involving a catheter, medical personnel may rely on x-ray, fluoroscopic, or other imaging to determine the location of the catheter within a patient's vascular system. In some embodiments, the outer surface 136 of catheter body 110 includes radiopaque (or otherwise visualizable) markings 180 that designate discrete distances or points along the length of catheter body 110 and/or a location of at least one of the ultrasound transducers 150. Radiopaque markings 180 are visible in an x-ray image and thus assist users in determining the precise location of the catheter body 110 and ultrasound transducers 150 within the patient during a procedure. Such markings are particularly useful in case backtracking procedures where fluoroscopy is required.

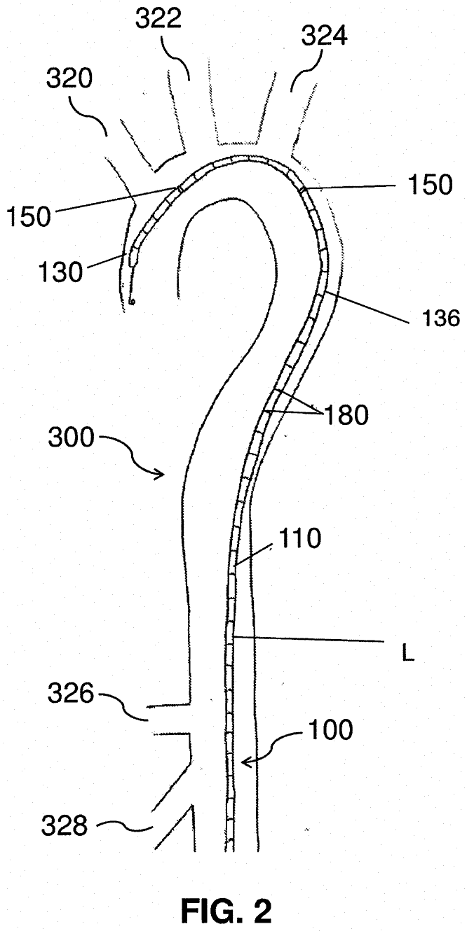

As an illustrative example, FIG. 2 provides a cutaway view of blood vessel 300 with IVUS catheter 100 positioned therein, according to various embodiments of the present disclosure. IVUS catheter 100 can be inserted into a blood vessel 300 (e.g., aorta), having accompanying branch vessels 320, 322, 324, 326, 328 stemming therefrom. Although not shown in FIG. 2, IVUS catheter 100 can be electronically connected to and in communication with user interface module 200, as described above in connection with FIG. 1.

As shown in FIG. 2, ultrasound transducers 150 can be positioned along a length of catheter body 110 proximate its distal end 130. In various embodiments, the positioning of ultrasound transducers 150 along catheter body 110 corresponds to an “area of interest” within the patient. The area of interest can be chosen based on diagnostic or therapeutic need. For example, the area of interest in FIG. 2 comprises the length of blood vessel 300 proximate branch vessels 320, 322, and 324. Accordingly, when IVUS catheter 100 is inserted into blood vessel 300, ultrasound transducers 150 can be positioned proximate the area of interest, (i.e., proximate branch vessels 320, 322, 324), and imagining of the region can be performed.

In certain embodiments, the area of interest can include diseased vessels, and data collected by IVUS catheter 100 can help guide therapy in real-time. For example, in FIG. 2, vessel 300 can present with atherosclerosis resulting in stenosis that can extend from branch vessels 320, 322, and 324 can present with atherosclerosis. A user can choose to image diseased vessels 320, 322, and 324 along a length or portion of blood vessel 300. The length or portion of blood vessel 300 imaged using IVUS catheter 100 can be referred to as the “active length.”

Additionally, for example, IVUS catheter 100 can be used to diagnose, characterize, and treat aortic aneurysms, aortic dissections, and venous stenosis. In other embodiments, IVUS catheter 100 can be used to aid in the administration of therapeutic interventions (treatments) such as balloon angioplasty or stent placement, thus reducing the use of fluoroscopy and contrast dye.

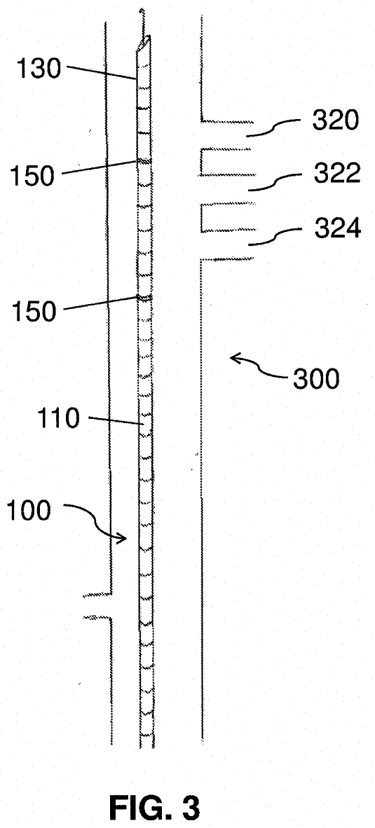

In certain embodiments, although IVUS catheter 100 can bend and turn during movement through various blood vessels, it can be beneficial to view images taken within the active length in a straight configuration. Accordingly, FIG. 3 illustrates IVUS catheter 100 and blood vessel 300 from FIG. 2, displayed in an uncurled configuration. The positioning of IVUS catheter 100 and ultrasound transducers 150 within blood vessel 300 and relative branch vessels 320, 322, and 324 is the same as in FIG. 2. However, imaging blood vessel 300 in an uncurled or straight configuration provides benefits to clinicians when displaying the images on monitor 210 of user interface module 200, as described above in relation to FIG. 1.

Monitor 210 of user interface module 200 can display a variety of images and information. To generate images from the ultrasound transducer 150 data, in some embodiments, imaging engine 220 can incorporate a software component with an algorithm to interpret, in combination, the data transmitted from the plurality of ultrasound transducers 150.

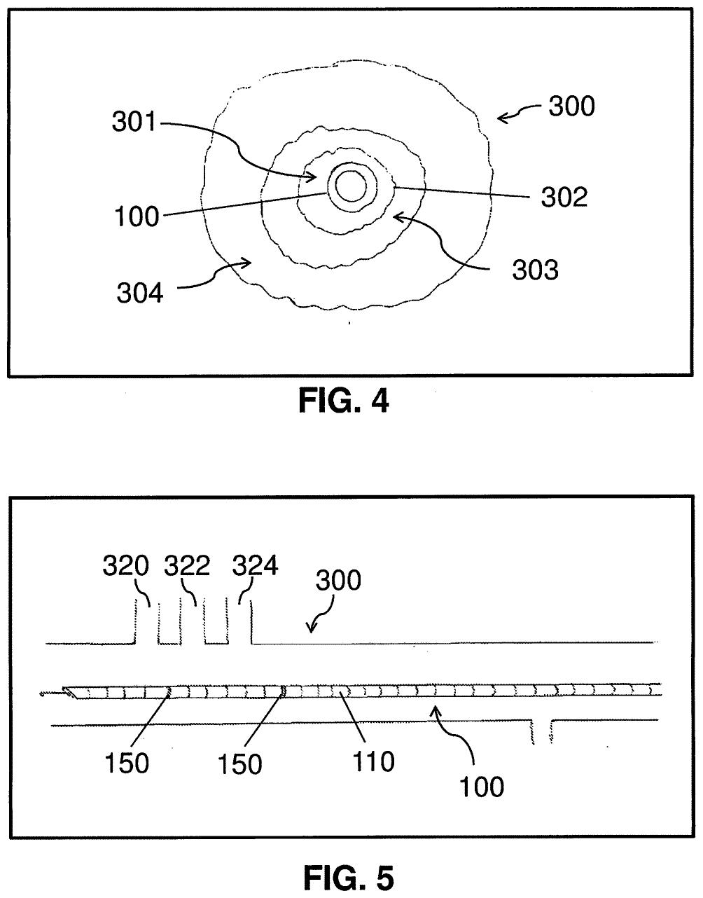

In certain embodiments, the software components can first generate two-dimensional (2-D) images of an axial view of the vessel of interest, which can then be displayed on monitor 210. For example, FIG. 4 illustrates one embodiment of a user interface layout displayed on monitor 210 depicting an ultrasound image of an axial cross-section of blood vessel 300. The axial ultrasound image illustrated in FIG. 4 can be generated by a single ultrasound transducer 150 and can provide a 6 to 10 cm wide, cross-sectional view of the tissues surrounding ultrasound transducer 150. In the center of the ultrasound image depicted in FIG. 4 is IVUS catheter 100, surrounded by lumen 301 of blood vessel 300, whose outer limits are defined by lumen boundary 302. In certain embodiments, plaque 303 is a circular mass surrounding lumen boundary 302. The intima, media and adventitia are displayed, in combination, as vessel wall 304.

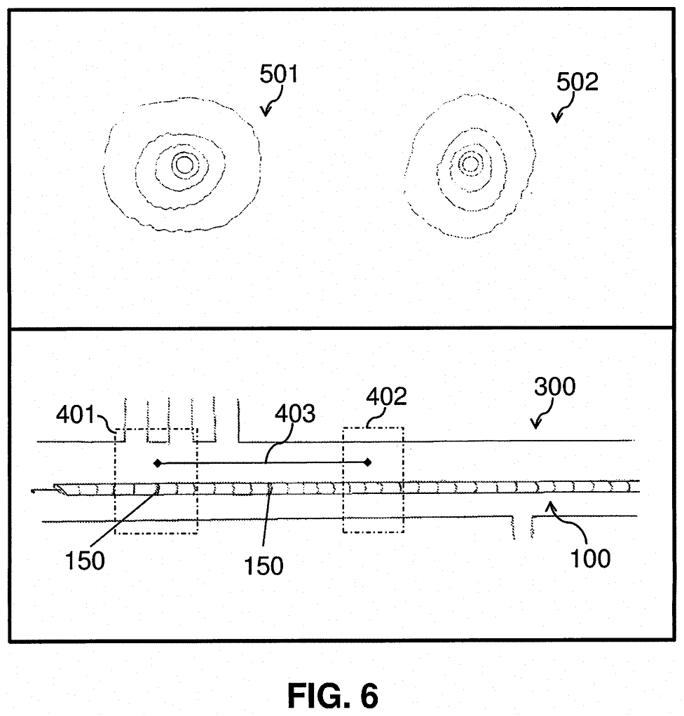

In some embodiments, the software components can then generate two-dimensional (2-D) ultrasound images of the longitudinal cross-section of the vessel of interest, which can then be displayed on monitor 210. For example, FIG. 5 illustrates one embodiment of a user interface layout displayed on monitor 210 depicting an ultrasound image of a longitudinal cross-section of blood vessel 300. IVUS catheter 100 is displayed in relation to blood vessel 300 and branch vessels 320, 322, and 324.

In some embodiments, user interface module 200 can display both axial images within and longitudinal images of the active length, simultaneously and in real-time. This advantage provides clinicians with near-instantaneous inputs relating to the anatomy of the area of interest. This feature can be beneficial for therapeutic interventions, such as stent placement, where knowledge of stent lumen diameter and the location of the stent relative to branch vessels is paramount to successful deployment.

According to various embodiments, during operation of treatment system 50, when initiating image acquisition, user interface module 200 can be adjusted or manipulated by the user. For example, in an exemplary embodiment, monitor 210 can include a touch screen, or user interface module can include a joy stick (not shown). Either a touch screen or joy stick can be used by a clinician to drag, drop, zoom, rotate, and/or mark the image. It can be appreciated that any suitable user interface module known in the art can be implemented used with treatment system 50 as described herein.

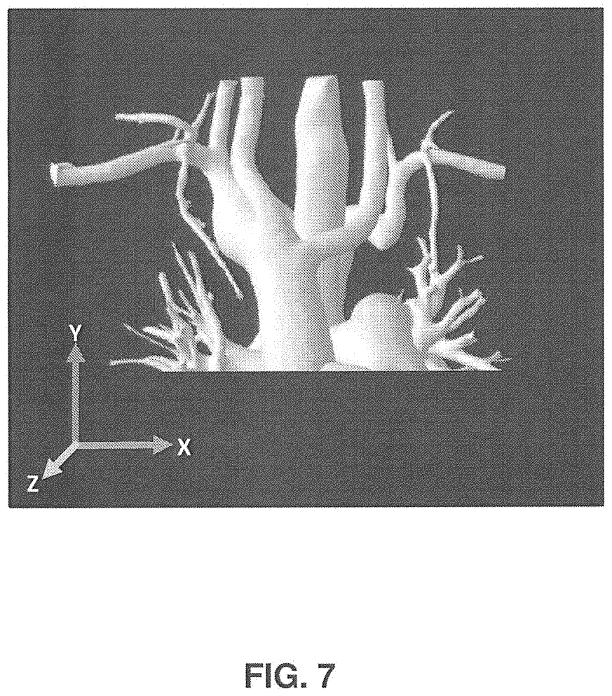

FIG. 6 illustrates one example of the functionality of a user interface layout displayed on monitor 210 depicting both axial (upper screen) and longitudinal (lower screen) cross-sections of blood vessel 300. In various embodiments, a user has the ability to mark one or more target areas, or areas of interest, on the longitudinal ultrasound cross-section using the user interface module. For example, according to various embodiments, a user can identify first target section 401 and a second target section 402 on monitor 210. A user can then command the imaging engine 220 to display axial, cross-sectional ultrasound images of each section on an upper screen of monitor 210. In various embodiments, the user interface also shows distance markers from the distal tip.

According to various embodiments, first cross-section 501 displays the cross-sectional image at the center of first target section 401. Additionally, second cross-section 502 displays the cross-sectional image at the center of second target section 402. In certain embodiments, a user can change the location of a target section, or select additional target sections within the active length of IVUS catheter 100. In some embodiments, a user can measure various distances or lengths within the longitudinal cross-sectional ultrasound image. For example, a user can draw length 403 on monitor 210 to determine the distance between the center points of first target section 401 and second target section 402. Additionally, a user can select various points on first cross-section 501 and second cross-section 502, instructing imaging engine 220 to output values or measurements indicative of various anatomical features, such as cross-sectional area, lumen diameters, plaque thicknesses and vessel wall thickness.

The above-mentioned functions of user interface module 200 enable users to guide therapeutic interventions or diagnostics. Further, treatment system 50 can provide the user with anatomical data of the blood vessels or vasculature of interest, including hemodynamics information (e.g., cardiac output, turbulence, velocity, etc.). The real-time output of treatment system 50 allows users to provide improved diagnostic and therapeutic treatments to a patient because it allows for faster clinician response times, more detailed information about branch vessels, and increased information about arterial geometries. The real-time imaging provided by treatment system 50 also allows for highly accurate targeting of treatment areas, precise selection of stent size and type, and accurate execution of various interventions, such as balloon angioplasty or stent delivery.

According to various embodiments, a secondary function of IVUS catheter 100 is to provide physiological monitoring of a patient during a medical procedure. In some embodiments, IVUS catheter 100 can further include pressure and temperature sensors to monitor pressure, such as blood pressure, and temperature within the patient. In an exemplary embodiment, the pressure and temperature sensors are MEMS sensors that can be fabricated on top of Application Specific Integrated Circuits (ASIC), which can reduce the overall cost of IVUS catheter 100.

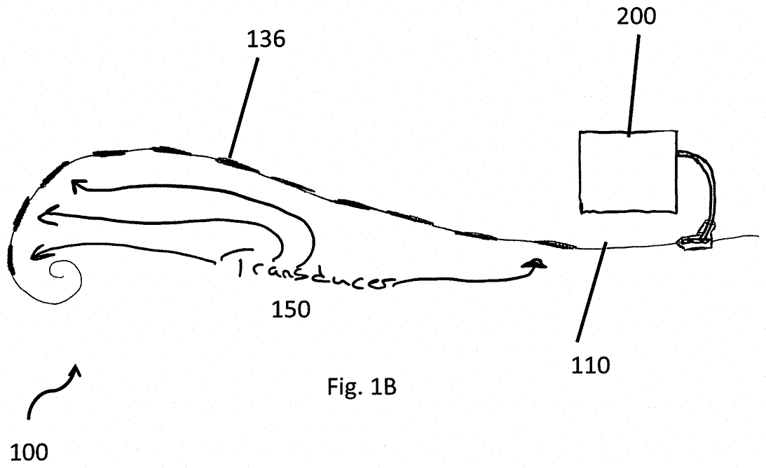

In some embodiments, when continuous data is transmitted to the imaging engine 300, the software components, using an algorithm, can be used to construct a 3-D image of the area of interest, as shown in FIG. 7. The 3-D image may then be displayed on monitor 210. Rendering a live, three-dimensional image allows for improved guidance during various vascular procedures. For example, live, 3-D images provided by systems of the present disclosure are helpful during an aneurysm repair, at least because they provide enhanced visualization of the contra lateral gate of the stent graft for cannulation. In some embodiments, imaging engine 220 interprets the data transmitted from both ultrasound transducers 150, merging multiple ultrasound images (e.g., phase array) to generate the 3-D image.

While the present disclosure recites many examples of IVUS technology, one skilled in the art will appreciate that the image processing techniques are not limited only to IVUS. Methods described in this application may also be utilized in other wave-based imaging techniques, for example phase-sensitive optical coherence tomography.

In certain embodiments, the positioning of multiple ultrasound transducers 150 on IVUS catheter 100 can provide clinical advantages over single-sensor designs. For example, multiple ultrasound transducers 150 on IVUS catheter 100, separated by certain distances, as disclosed herein, can be used to image, sense or otherwise monitor blood vessels, including branch vessels, simultaneously within different regions of the vascular system. For example, in certain embodiments, once IVUS catheter 100 is fully inserted, at least one ultrasound transducers 150 can be positioned to capture an image in the pulmonary artery and at least one other transducer 150 can be positioned to capture an image in the right ventricle of a human heart. In this configuration, the IVUS catheter 100 can be used to measure or visually inspect critical physiologic abnormalities present in advanced heart failure.

In some embodiments, multiple positions and configurations of the ultrasound transducers 150 throughout the IVUS catheter 100 can be provided to measure, diagnose, or image multiple regions of the cardiovascular system. In some embodiments, the multiple ultrasound transducers or sensors 150 can share a wiring lumen. In some embodiments, the ultrasound transducers 150 described herein can be arranged in a helical pattern along the distal end of IVUS catheter 100 or can be arranged with a pitch.

In various embodiments, image data can be captured using a timing delay. For example, the treatment system 50 can implement a time delay when sensing the electrical signal from the plurality of transducers 150 of IVUS catheter 110 depending on the depth of the electrical signals. Additionally, software coding can be used to modulate the acoustic wave signals described above. For example, software can be used to change the frequency per transducer 150 (or group of ultrasound transducers 150) in order to make the signal specific to a group (e.g., radio signals).

Therefore, the present invention generally provides for a method of using an IVUS catheter 100 for imaging tissue in real-time, by inserting an IVUS catheter 100 in the body of an individual, wherein the IVUS catheter 100 includes a catheter body 110 having a proximal end 120, a distal end 130 opposite thereto, and an outer surface 136 having a plurality of ultrasound transducers 150, and providing real-time and simultaneous imaging of tissue with the plurality of ultrasound transducers 150.

The present invention also provides several methods of specific applications, further described in the examples below.

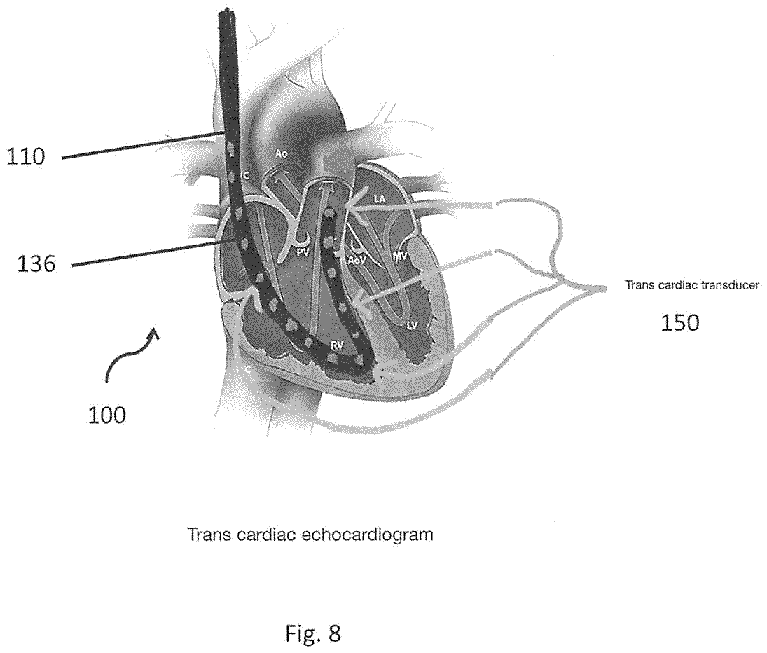

The present invention provides for a method of performing trans cardiac echocardiography by inserting an IVUS catheter or wire 100 in the body of an individual through jugular femoral or subclavian venous access and then subsequently guiding the device into the patient's right atrium, right ventricle or pulmonary artery, wherein the IVUS catheter or wire 100 includes a catheter body 110 having a proximal end 120, a distal end 130 opposite thereto, and an outer surface 136 having a plurality of ultrasound transducers 150, and providing real-time and simultaneous imaging of the individual's heart with the plurality of ultrasound transducers 150 and allowing for imaging of the cardiac chambers in real time, including doppler flow acquisition and analysis, color flow acquisition and analysis. An example of this use is shown in FIG. 8.

The present invention provides for a method of performing trans esophageal echocardiography by inserting an IVUS catheter or wire 100 in the body of an individual into an esophagus, wherein the IVUS catheter 100 includes a catheter body 110 having a proximal end 120, a distal end 130 opposite thereto, and an outer surface 136 having a plurality of ultrasound transducers 150, and providing real-time and simultaneous imaging of the individual's heart with the plurality of ultrasound transducers. Preferably, imaging is provided along the entire length of the IVUS catheter or wire 100.

The present invention provides for a method of imaging the bladder and prostate by inserting an IVUS catheter or wire 100 in the body of an individual transurethrally, wherein the IVUS catheter or wire 100 includes a catheter body 110 having a proximal end 120, a distal end 130 opposite thereto, and an outer surface 136 having a plurality of ultrasound transducers 150, and providing real-time and simultaneous imaging of the individual's bladder and prostate with the plurality of ultrasound transducers 150. Preferably, imaging is provided along the entire length of the IVUS catheter or wire 100.

The present invention also provides for a method of imaging the gastrointestinal system by inserting an IVUS catheter or wire 100 in the body of an individual into the gastrointestinal system, wherein the IVUS catheter or wire 100 includes a catheter body 110 having a proximal end 120, a distal end 130 opposite thereto, and an outer surface 136 having a plurality of ultrasound transducers 150, and providing real-time and simultaneous imaging of the individual's gastrointestinal system with the plurality of ultrasound transducers 150. Preferably, imaging is provided along the entire length of the IVUS catheter or wire 100.

Generally, the IVUS catheter 100 of the present disclosure provides significant benefits over traditional IVUS catheters due to the arrangement of multiple transducers 150 placed along a distal end 130 length of the IVUS catheter 100 and in conjunction with unique software to generate a single visual output (e.g., merging of ultrasound images from various sources, phased array). Additional embodiments and configurations of the present disclosure will be obvious to a person of ordinary skill in the art.

The use of IVUS catheters in therapeutic applications provides medical professionals greater detail about a blood vessel, while further reducing the need to expose the patient to radiation or nephrotoxic chemicals, as well as limiting the radiation exposure to the operator and staff. Unlike angiography, IVUS catheters provide users with more detail about plaque formation and morphology within the vessel, for example, plaque density and thickness. IVUS catheters also offer greater detail with more accuracy regarding the anatomy of the vessels, such as the location of vessel branches. Additionally, IVUS catheter imaging can be used to construct three-dimensional (3-D) images or representations of the vessel and surrounding tissues. Accordingly, the present disclosure relates to an IVUS catheter system that provides advantages over existing devices.

One skilled in the art will appreciate that the techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

Further, the techniques described in this disclosure can also be embodied or encoded in a non-transitory computer-readable medium, such as a computer-readable storage medium, containing instructions. Instructions embedded or encoded in a computer-readable storage medium may cause a programmable processor, or other processor, to perform the method, e.g., when the instructions are executed. Non-transitory computer readable storage media may include volatile and/or non-volatile memory forms including, e.g., random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE 1

Use of the IVUS catheter for trans cardiac echocardiography.

The catheter is positioned through jugular venous access and wired into position into the pulmonary artery. The same intravascular ultrasound design from IVUS live can be utilized, with transducers that are utilizing a lower frequency to obtain images of the all the chambers of the heart. This can additionally be utilized to interpret filling ejection fraction of the right ventricle (RV), left ventricle (LV) and filling pressures of the various chambers as well. This can be left in place continuously in lieu of a swan ganz catheter to give real time hemodynamic information about what is actually occurring In the function of a sick heart and used to guide therapy. The software required to analyze interpret and present this information can be different in comparison to the IVUS catheter. This approach to cardiac imaging allows enough information to build a three dimensional model with a real time ability to view the function of the heart.

### EXAMPLE 2

Use of the IVUS catheter for trans esophageal echocardiography.

The catheter is inserted via a nasogastric or oropharyngeal method and subsequently passed into the esophagus to obtain transesophageal echocardiograms. With multiple ultrasound transducers, the need to continuously change the positioning of the current probes is no longer necessary and can provide views of all the chambers of the heart simultaneously. This application can require a different model then the one used for intravascular ultrasound as the ultrasound requirements are different. It can require lower frequency ultrasound transducers and have deeper penetration as the images are being obtained through several different structures, but the concept of having multiple probes on the catheter a set intervals, and being able to merge the images that are obtained together is the same. This approach to cardiac imaging allows enough information to build a three dimensional model with a real time ability to view the function of the heart.

### EXAMPLE 3

Genitourinary applications.

The catheter is inserted transurethral and utilized to image the bladder and prostate, giving information such as prostate size, location of masses in the bladder and prostate as well as depth of invasion. This can also be utilized to guide procedures such as transurethral resection of the prostate, with more accurate information regarding the depth of resection, thereby allowing the proceduralist to remove more tissue safely.

### EXAMPLE 4

Gastrointestinal applications.

The catheter can have a similar design to the GU catheter but a larger size to allow for better visualization of structures in the esophagus and rectum and to help guide therapy such as resection of mass.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

What is claimed is:

1. An intravascular ultrasound (NUS) catheter or wire for imaging body tissue, comprising a catheter body having a proximal end, a distal end opposite thereto, and an outer surface having a plurality of ultrasound transducers operatively attached thereto that provide real-time and continuous imaging of said body tissue along an entire length of said body tissue.

2. The IVUS catheter or wire of claim 1, wherein said catheter body is made from a material chosen from the group consisting of biocompatible polymers, elastomer, silicon, nylon, and combinations thereof.

3. The IVUS catheter or wire of claim 1, wherein said outer surface is coated with a coating chosen from the group consisting of a hydrophilic coating, a heparin-based coating, and an anti-thrombotic coating.

4. The IVUS catheter or wire of claim 1, wherein said catheter body further includes at least one lumen extending at least partially along a length of said catheter body.

5. The IVUS catheter or wire of claim 4, wherein at least one opening extends between said at least one lumen and said outer surface for fluid communication to vasculature of a patient.

6. The IVUS catheter or wire of claim 1, wherein said distal end includes an atraumatic tip.

7. The IVUS catheter or wire of claim 1, further including a connector hub operatively attached at said proximal end and providing at least one access line.

8. The IVUS catheter or wire of claim 1, wherein 2-3000 ultrasound transducers are provided.

9. The IVUS catheter or wire of claim 8, wherein said ultrasound transducers are a sensor chosen from the group consisting of microelectromechanical sensors, capacitive sensors, piezoelectric sensors, and combinations thereof.

10. The IVUS catheter or wire of claim 9, wherein groups of ultrasound transducers are positioned at discrete locations along a length of said catheter body.

11. The IVUS catheter or wire of claim 1, further in electronic communication with a user interface module including a monitor and imaging engine.

12. The IVUS catheter or wire of claim 11, wherein said user interface module sends electrical signals to said ultrasound transducers continuously and generates real-time, continuous imaging.

13. The IVUS catheter or wire of claim 1, wherein said outer surface further includes radiopaque markings designating a point chosen from the group consisting of distances along a length of said catheter body, a location of at least one ultrasound transducer, and combinations thereof.

14. The IVUS catheter or wire of claim 1, further including pressure and temperature sensors.

15. The IVUS catheter or wire of claim 1, wherein said catheter body has a length of 10-150 cm and a diameter of 3-12 Fr.

16. The IVUS catheter or wire of claim 1, wherein said ultrasound transducers have a frequency of 20-100 MHz.

17. A method of using an intravascular ultrasound (IVUS) catheter or wire for imaging tissue in real-time, including the steps of:
inserting an IVUS catheter or wire in the body of an individual, wherein the IVUS catheter or wire includes a catheter body having a proximal end, a distal end opposite thereto, and an outer surface having a plurality of ultrasound transducers; and
providing real-time and simultaneous imaging of tissue with the plurality of ultrasound transducers along an entire length of the tissue.

18. The method of claim 17, wherein said providing step is further defined as displaying images of the tissue on a monitor of a user interface module in electronic communication with the IVUS catheter.

19. The method of claim 18, further including the step of the user interface module sending electrical signals to the ultrasound transducers continuously and generating real-time, continuous imaging.

20. The method of claim 19, wherein said providing step includes the ultrasound transducer converting the received electrical signal into an acoustic energy pulse or pressure wave emitted in a 360° manner about the catheter body.

21. The method of claim 20, wherein said providing step is further defined as reflecting emitted signals off of tissue, the ultrasound transducers acquiring the reflected acoustic signals back into an electrical signal, which is transmitted back to the user interface module.

22. The method of claim 19, further including the step of transmitting acoustic data between the ultrasound transducers and the user interface module.

23. The method of claim 17, wherein said providing step is further defined as generating and displaying a two-dimensional image of an axial view of a vessel of interest, and generating and displaying two-dimensional ultrasound images of a longitudinal cross-section of the vessel of interest.

24. The method of claim 23, wherein said generating and displaying steps are performed simultaneously and in real-time.

25. The method of claim 24, further including the step of providing three-dimensional images of tissue.

26. The method of claim 17, further including the step of imaging tissue chosen from the group consisting of blood vessels, arterial plaques, and branch vessels.

27. The method of claim 17, further including the step of imaging when the catheter body is in a straight configuration.

28. The method of claim 17, further including the step of using the IVUS catheter for a procedure chosen from the group consisting of diagnosing a disease, characterizing a disease, and treating a disease.

29. The method of claim 18, further including the step of adjusting the user interface module by an action chosen from the group consisting of dragging, dropping, zooming, rotating, marking an image, and combinations thereof.

30. The method of claim 29, further including the step of marking at least one target area on the image of tissue.

31. The method of claim 17, further including the step of providing a user with anatomical data of blood vessels or vasculature.

32. The method of claim 31, further including the step of providing a user with hemodynamic information.

33 The method of claim 17, further including the step of providing physiological monitoring of an individual during a medical procedure.

34. The method of claim 17, further including the steps of capturing an image in a pulmonary artery with at least one ultrasound transducer, and capturing an image in a right ventricle of a heart with at least one ultrasound transducer.

35. A method of performing trans cardiac echocardiography, including the steps of:
inserting an IVUS catheter or wire in the body of an individual through jugular, femoral, or subclavian venous access and then subsequently guiding the device into the individual's right atrium, right ventricle or pulmonary artery, wherein the IVUS catheter or wire includes a catheter body having a proximal end, a distal end opposite thereto, and an outer surface having a plurality of ultrasound transducers; and
providing real-time and simultaneous imaging of the individual's heart with the plurality of ultrasound transducers and allowing for imaging of cardiac chambers in real time.

36. A method of performing trans esophageal echocardiography, including the steps of:
inserting an IVUS catheter or wire in the body of an individual into an esophagus, wherein the IVUS catheter or wire includes a catheter body having a proximal end, a distal end opposite thereto, and an outer surface having a plurality of ultrasound transducers; and
providing real-time and simultaneous imaging of the individual's heart with the plurality of ultrasound transducers.

37. A method of imaging the bladder and prostate, including the steps of:
inserting an IVUS catheter or wire in the body of an individual transurethrally, wherein the IVUS catheter or wire includes a catheter body having a proximal end, a distal end opposite thereto, and an outer surface having a plurality of ultrasound transducers; and
providing real-time and simultaneous imaging of the individual's bladder and prostate with the plurality of ultrasound transducers.

38. A method of imaging the gastrointestinal system, including the steps of:
inserting an IVUS catheter or wire in the body of an individual into the gastrointestinal system, wherein the IVUS catheter or wire includes a catheter body having a proximal end, a distal end opposite thereto, and an outer surface having a plurality of ultrasound transducers; and
providing real-time and simultaneous imaging of the individual's gastrointestinal system with the plurality of ultrasound transducers.
